(19) United States
(12) Patent Application Publication
(10) Pub. No.: US 2004/0209340 A1
(43) Pub. Date: Oct. 21, 2004

(54) Enzymatic degradation chains

(76) Inventors: Johannes Georg Schindler, Marburg/Lahn (DE); Hans-Willi Kling, Wuppertal (DE); Jens Seeman, Erkrath (DE)

Correspondence Address: COGNIS CORPORATION, PATENT DEPARTMENT, 300 BROOKSIDE AVENUE, AMBLER, PA, 19002, US

(21) Appl. No.: 10/474,652

(22) PCT Filed: Apr. 11, 2002
(86) PCT No.: PCT/EP02/04043

(30) Foreign Application Priority Data
Apr. 14, 2001 (DE) 101 18 554.5
Apr. 14, 2001 (DE) 101 18 553.7

(51) Int. Cl. C12N 9/00

(52) U.S. Cl. 435/183

(57) ABSTRACT

A process for reacting a target molecule with at least two different enzymes involving the steps of: (a) providing at least one target molecule; (b) providing at least one reaction zone; (c) providing at least two different enzymes, present in the reaction zone, wherein at least one of the enzymes is immobilized therein; (d) introducing the target molecule into the reaction zone; and (e) reacting the target molecule, with the enzymes, in the reaction zone.

This invention relates to enzymatic systems containing enzyme degradation chains of enzymes of various types, preferably in the immobilized state, and to their use, more particularly in bioreactors, biosensors and chromatographic systems.

In applied microbiology, more particularly in biotechnology, it is known that enzymes, enzyme-producing microorganisms or cells can be fixed to certain carriers, particularly if they are used as biocatalysts. This process is known generally as immobilization.

Since native enzymes are reduced in their activity by biological, chemical or physical effects during storage or in “one-off” batch applications, there is a need to stabilize the enzymes in view of their high production costs. Through immobilization, the enzymes become reusable. After use, the enzymes are easy to remove. In this way, they can be used in high local -concentrations and in continuous throughflow. The substrate specificity and the specificity of the reaction and also the reactivity of the enzymes should not be lost as a result of immobilization.

In general, enzymes can be immobilized by three basic methods, namely: first, immobilization by crosslinking, second immobilization by binding to a carrier and, third, immobilization by enclosure.

Where immobilization is carried out by crosslinking, the crosslinked enzymes obtained are fixed to one another without any effect on their activity. However, the enzymes are no longer soluble. Crosslinking is carried out, for example, with glutardialdehyde.

Where enzymes are immobilized by binding to a carrier, binding may be carried out by adsorption, ionic bonding or covalent bonding. Binding to the carrier may even take place within the original microbial cell. The enzyme is not influenced in its activity as a result of fixing and may be repeatedly or continuously used fixed to a carrier.

In immobilization by enclosure, the enzyme is generally enclosed between semipermeable membranes and/or gels, microcapsules or fibers. The encapsulated enzymes are separated from the surrounding substrate and product solution, for example, by a semipermeable membrane. Even cells can be encapsulated. The enzyme is not influenced in its activity by fixing in space.

Immobilized enzymes, enzyme-producing microorganisms or cells are used in particular in biotechnological processes. The first industrial processes using immobilized cells were empirically optimized and are still in use today, as for example wastewater treatment in a bacteria bed. Another fairly old process is the production of vinegar by the generator process. In the food industry, the use of cells containing glucose isomerase is the most important process for the production of fructose-containing syrup. Glucose amylase for the production of glucose by the starch process is also used in immobilized form. The splitting of lactose into glucose and galactose using immobilized β-galactosidase from yeasts is another standard process. Other industrial processes using immobilized enzymes are applied in the production of amino acids, in the splitting of penicillin G into 6-aminopenicillic acid and in the production of ethanol with growing immobilized cells of Saccharomyces sp.

Immobilized enzyme and cell systems are used not only in biotechnological production processes, but also in analysis, for example in so-called biosensors. The principle of analysis using immobilized systems is based on the reaction of a substrate to be determined by an immobilized system, the changes in the concentrations of product, substrate and co-substrate being able to be followed, for example by several coupled methods (for example enzyme electrodes).

Often, however, the degradation or the reaction of molecules is not effected by a single enzyme, but along a so-called enzyme degradation chain of several enzymes, i.e. the reaction is enzyme-catalyzed by different enzymes in several stages. Occasionally, the parallel determination of several substances by several different enzymes or by enzyme (degradation) chains is also necessary. In many cases, however, a repeatedly enzyme-catalyzed reaction sequence such as this can only rarely be carried out in conventional biosensors and bioreactors because such bioreactors and biosensors preferably comprise one type of enzyme.

The problem addressed by the present invention was to provide a system with which it would be possible to enzymatically catalyze even those processes that take place enzymatically in several stages, i.e. where the molecules, more particularly biomolecules or technical molecules, are degraded or reacted to end products in several enzyme-catalyzed stages, the reaction having to take place within only a single enzymatic system, but with different enzymes.

Another problem addressed by the invention was to provide a system with which several substances could also be reacted in parallel by several different enzymes or by enzyme (degradation) chains.

Such a system would be particularly suitable for use in bioreactors, biosensors and chromatographic systems.

It has now surprisingly been found that the problem stated above can be solved by combination of at least two different enzymes, i.e. at least two different types of enzymes, of which each may preferably be present in immobilized form.

Accordingly, the present invention relates to an enzymatic system comprising an enzyme degradation chain which in turn comprises at least two different enzymes, i.e. enzymes of different types.

The at least two enzymes of the enzyme (degradation) chain are coordinated with one another in such a way that they degrade, more particularly selectively or substantially selectively, at least one particular molecule also known as the “target molecule”.

In a first embodiment of the present invention, the enzymes of the enzyme (degradation) chain may be coordinated with one another in such a way that they degrade, more particularly selectively or substantially selectively, at least one non-naturally occurring molecule, more particularly a technical molecule. In this embodiment, the enzymes of the enzyme (degradation) chain may be coordinated with one another, for example, in such a way that they degrade the non-naturally occurring molecule, more particularly the technical molecule, according to a non-naturally occurring metabolism, i.e. form a non-naturally occurring metabolism chain. Alternatively, however, the enzymes of the enzyme (degradation) chain may also be coordinated with one another in such a way that they degrade the non-naturally occurring molecule, more particularly the technical molecule, according to a naturally occurring metabolism, i.e. form a naturally occurring metabolism chain which—occurring naturally—regulates the degradation of other, i.e. naturally occurring, molecules.

In a second embodiment of the present invention, the enzymes of the enzyme (degradation) chain may be coordinated with one another in such a way that they degrade, more particularly selectively or substantially selectively, at least one naturally occurring molecule according to a non-naturally occurring metabolism. The naturally occurring molecule may be, for example, a natural substance.

The enzymatic system according to the invention is particularly suitable for the consecutive and/or multistage degradation of molecules. The degradation process is preferably selective or substantially selective in relation to the molecule(s) to be degraded.

In addition, the enzymatic system according to the invention may be used in particular for the catalysis of reactions catalyzed by enzymes in several stages.

The enzymatic system according to the invention is equally suitable for the parallel reaction of different molecules with different enzymes.

In one particular embodiment of the present invention, at least one of the enzymes of the enzyme (degradation) chain is present in immobilized form in the enzymatic system according to the invention. In this embodiment, all the enzymes of the enzyme (degradation) chain may preferably be present in immobilized form.

According to the invention, the immobilization of the enzymes may be carried out by any of the known processes described above, the reactivity of the enzymes thus immobilized remaining at least substantially intact.

Thus, the immobilization of at least one of the enzymes of the enzymatic system according to the invention may be carried out, for example, by crosslinking.

In addition, the immobilization of at least one of the enzymes of the enzymatic system according to the invention may be carried out by enclosure or encapsulation, more particularly by enclosure or encapsulation in a semipermeable membrane or between several semipermeable membranes or even between one and/or several semipermeable membranes and, finally, an ion-impermeable but gas-permeable membrane, for example of PTFE or silicone rubber.

The immobilization of at least one of the enzymes of the enzymatic system according to the invention may also be carried out by binding to a suitable carrier. In this case, binding can be achieved by adsorption, ionic bonding and/or covalent bonding. For example, the enzyme may be immobilized by binding to a suitable, preferably chemically inert carrier which may have been activated beforehand by plasmachemical surface modification.

Finally, the enzyme(s) of the enzymatic system according to the invention may also be immobilized by the process described in German patent application DE 101 18 553.7 of which the entire disclosure is hereby expressly included by reference.

This process comprises the following steps:

(a) activating the chemically inert carrier surface by modification of that surface by plasmachemical methods, the activation of the chemically inert carrier surface comprising in particular the functionalization of that surface and preferably being carried out by at least one suitable functional group reactive to the enzyme(s) to be bound being directly applied to the chemically inert carrier surface under plasmachemical conditions, the functional group preferably being a carboxyl, amino, hydroxy and/or thio group, optionally in activated, more particularly protonated or deprotonated, form; then

(b) binding the enzyme(s) to be immobilized, optionally after it/they has/have been converted into an activated or fixable state, to the carrier surface activated in step (a); and finally

(c) optionally crosslinking the enzymes bound to the carrier surface in step (b),

process steps (b) and (c) in one particular embodiment optionally being carried out in combination, more particularly at the same time, and/or the immobilization optionally being carried out in layers, more particularly with glutardialdehyde.

Accordingly, in one particular embodiment, the present invention relates to an enzymatic system in which at least one of the enzymes and preferably all the enzymes of the enzyme (degradation) chain is/are immobilized by fixing and/or binding to a chemically inert, plasmachemically activated and/or functionalized carrier surface by the process described above.

In a preferred embodiment, the activation of the chemically inert carrier surface by plasmachemical methods in step (a) of the process described above is carried out selectively on the surface only so that the bulk properties of the chemically inert carrier surface remain otherwise intact, the activation of the chemically inert carrier surface in step (a) of the process described above taking place in a reactive plasma, more particularly a high-frequency plasma. The chemically inert carrier surface may comprise in particular noble metals, such as in particular platinum and alloys thereof, stainless steel or polyhalogenated polymers, more particularly polyhalogenated polymeric hydrocarbons such as, in particular, polytetrafluoroethylene or polyvinyl chloride, or even cellulose acetate or combinations of these materials. PTFE (Teflon®) and cellulose acetate membranes, for example, are particularly suitable.

The binding or coupling of the enzymes to be immobilized to the plasmachemically modified carrier surface in step (b) of the above-described process may be carried out in particular by binding or coupling of the enzyme(s) via the reactive functional groups applied in step (a), the enzyme(s) being bound to the reactive functional groups applied to the carrier surface either directly or indirectly via a suitable linker.

In one particular embodiment, the present invention relates to an enzymatic system in which at least one of the enzymes and preferably all the enzymes of the enzyme (degradation) chain is/are immobilized by fixing and/or binding to a chemically inert, plasmachemically activated and/or functionalized carrier surface. The enzyme(s) may be directly or indirectly fixed, more particularly bound or coupled, to a chemically inert carrier surface. In one particular embodiment, the enzyme(s) may be bound or coupled via suitable reactive functional groups applied to the chemically inert carrier surface.

The enzymes of the enzymatic system according to the invention may be selected in particular from the group of oxidoreductases, transferases, hydrolases (for example esterases, such as lipases), lyases, isomerases and ligases (synthetases) and mixtures or combinations thereof with one another.

In the enzymatic system according to the invention, the at least two enzymes are disposed either in a single reaction zone or in separate successive, sequentially arranged reaction zones which then together form the enzymatic system. In other words, in the enzymatic system according to the invention, the enzymes of the enzyme (degradation) chain may be arranged within a unit, preferably within a reaction zone, a compartment or a cell, more particularly a measuring cell, or even in separate, more particularly successive, units, preferably in separate successive reaction zones, compartments or cells, more particularly measuring cells, which together form the enzymatic system. However, measuring cells may also be arranged in parallel for the purpose of differential measurement.

In one particular embodiment of the present invention, the enzymatic system according to the invention contains amyloglucosidase and glucoseoxidase, optionally in combination with mutarotase and/or α-glucosidase (maltase). A system such as this is particularly suitable for the degradation of nonionic surfactants, more particularly alkyl polyglucosides.

The enzymatic system according to the invention may be used for many purposes, more particularly in biosensors, bioreactors or chromatographic systems (for example chromatographic columns). Accordingly, the present invention also relates to biosensors, bioreactors and chromatographic systems which comprise the enzymatic system according to the invention.

As described above, the enzymatic systems according to the invention may be used in biosensors. At least two different types of immobilized enzymes used in an enzyme chain or enzyme degradation chain are combined with one another. The different enzymes may either be present in a single reaction system (for example in a measuring cell) or may be sequentially arranged one behind the other (for example in successive measuring cells) which then together form the enzymatic system according to the invention. In this way, it is possible, for example, to determine several substances “in parallel” by several enzymes (or enzyme chains) in a measuring cell or sequentially to degrade a starting material (analyte) in several measuring cells arranged in tandem with simultaneous electroanalysis in one and/or more measuring cells.

In the context of the invention, “biosensors” are understood to be sensors containing a bioactive component based on the coupling of biomolecules which, as receptors in the broadest sense, specifically recognize analytes with physicochemical transductors which convert a biologically produced signal (for example oxygen concentration, pH, dye, etc.) into electrical measuring signals.

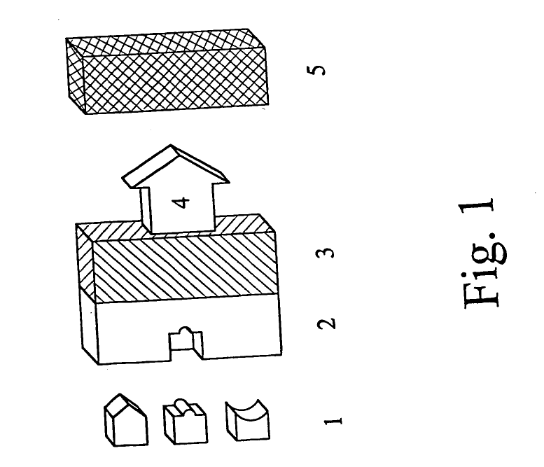

FIG. 1 schematically illustrates the typical construction of a biosensor for specifically recognizing an analyte 1, the biosensor comprising a receptor 2 and a transductor 3 which converts the biological signal produced by the receptor 2 into an electronic signal 4 which is transmitted to an electronic circuit 5.

Various biomolecules, more particularly enzymes, may be used for specific recognition. The transductors used may be potentiometric sensors, amperometric electrodes, piezoelectric sensors, thermistors or optoelectronic sensors. There are in particular two basic types of biosensors, depending on the reaction or interaction of the analyte with the receptor. On the one hand, there are bioaffinity sensors, which use the change in electron density occurring during complexing, and on the other hand metabolism sensors which are based on the specific recognition and reaction of substrates.

Biosensors are used—particularly in the form of enzyme electrodes—in healthcare, for monitoring biotechnological processes, in the food industry or in environmental protection. Various systems, for example glucose, galactose, lactose, ethanol, lactic acid or uric acid, can be analyzed with biosensors.

Where the enzymatic system according to the invention is used in biosensors, for example, the various enzyme molecules may be introduced for immobilization either into polymeric matrixes (such as, for example, PVC, gels, graphites or zeolites) or between films (for example cellulose acetate). In addition, in the case of sensors which are based, for example, on the enzymatic production or the consumption of oxygen and which have a chemically inert membrane (for example a Teflon® membrane), this membrane may be used for binding the various enzymes. The biosensors according to the invention may be produced, for example, as described in the above-cited German patent application DE 101 18 553.7 of which the entire disclosure is hereby included by reference.

The biosensors according to the invention are suitable, for example, for the production of microelectrode (arrays) for small volumes and high sample throughputs (for example for combinatorial use).

Examples of applications for the biosensors according to the invention include the analytical determination of surfactants, more especially ionic surfactants, such as nonionic surfactants (for example alkyl polyglucosides), polyaspartic acid and fatty alcohol derivatives.

In one particular embodiment, the present invention relates to a biosensor, more particularly for the qualitative and/or quantitative determination of nonionic surfactants, preferably alkyl polyglucosides, characterized in that the biosensor comprises as its enzyme (degradation) chain an enzymatic system which contains amyloglucosidase and glucose oxidase, optionally in combination with mutarotase and/or α-glucosidase (maltase), as enzymes. The enzymes mentioned are present in particular in immobilized form, preferably by binding to a chemically inert surface and/or membrane, preferably to a polytetrafluoroethylene or cellulose acetate membrane.

As described above, the enzymatic system according to the invention may also be used in bioreactors.

In the context of the invention, “bioreactors” are understood to be the physical container in which biological conversions are carried out, more particularly with enzymes.

The immobilized enzymes of the enzymatic system according to the invention may be applied, for example, to the wall surfaces of the bioreactor or—particularly in the case of fixed-bed reactors—may be bound to the stationary carrier material or bulk material. Particulars can be found in the above-cited German patent application DE 101 18 553.7 of which the entire disclosure is hereby included by reference in the present application. It is possible in this way to construct a more efficient generation of reactors where the enzymes do not have to be separated from the reaction solution. Reactor surfaces suitable for the purposes of the invention may consist of metal (for example noble metal, such as platinum) or may be coated with any of the polymers typically used for the production of reactors (for example Teflon®).

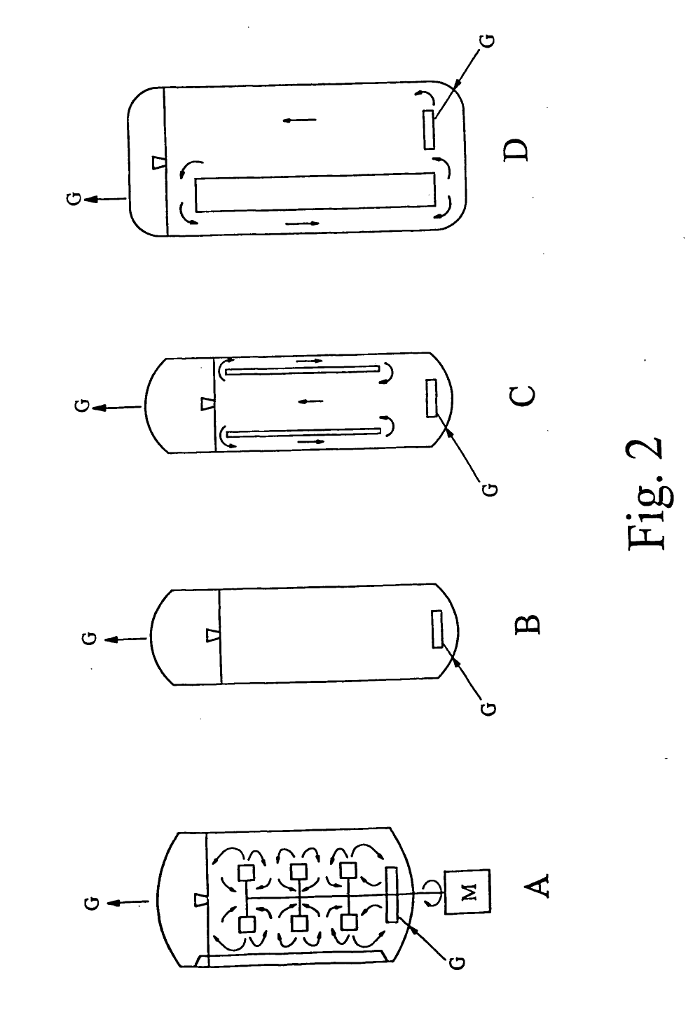

By way of example, FIG. 2 schematically illustrates various types of known bioreactors.

FIG. 2A shows a stirred tank reactor in which energy is introduced by mechanically moved units. The letter G denotes the gas stream and the letter M denotes the mechanical drive system (for example motor). By virtue of their versatility, stirred tank reactors are the most commonly used.

FIG. 2B shows a bubble column reactor where mixing is effected by the introduction of air or another gas. The letter G denotes the gas stream.

FIG. 2C shows a so-called airlift fermenter with internal throughflow, the circulation of liquid and mixing generally being achieved by the introduction of air or another gas. The letter G denotes the gas stream.

FIG. 2D shows a so-called airlift fermenter with external throughflow, the circulation of liquid and mixing generally being achieved by the introduction of air or another gas. The letter G denotes the gas stream.

The enzymatic system according to the invention may be used in the known types of bioreactor described above, for example by modification of the wall surface (for example by coupling of the enzymes onto the chemically inert reactor walls) or—in the case of fixed-bed bioreactors—by binding of the enzymes to the stationary carrier material or bulk material.

Where the enzymatic system according to the invention is used in bioreactors, more particularly for modifying the wall surface or in the binding of the enzymes to the stationary carrier material or bulk material, the at least two different types of enzymes may be combined with one another in such a way, i.e. the enzyme chains or enzyme degradation chains may be used in such a way, that either the various enzymes are present in a single reaction zone or are arranged sequentially one behind the other (for example in successive reaction zones). This enables enzyme-catalyzed syntheses and processes to be carried out, more particularly in several stages, or various starting materials to be simultaneously reacted “in parallel” by various enzymes.

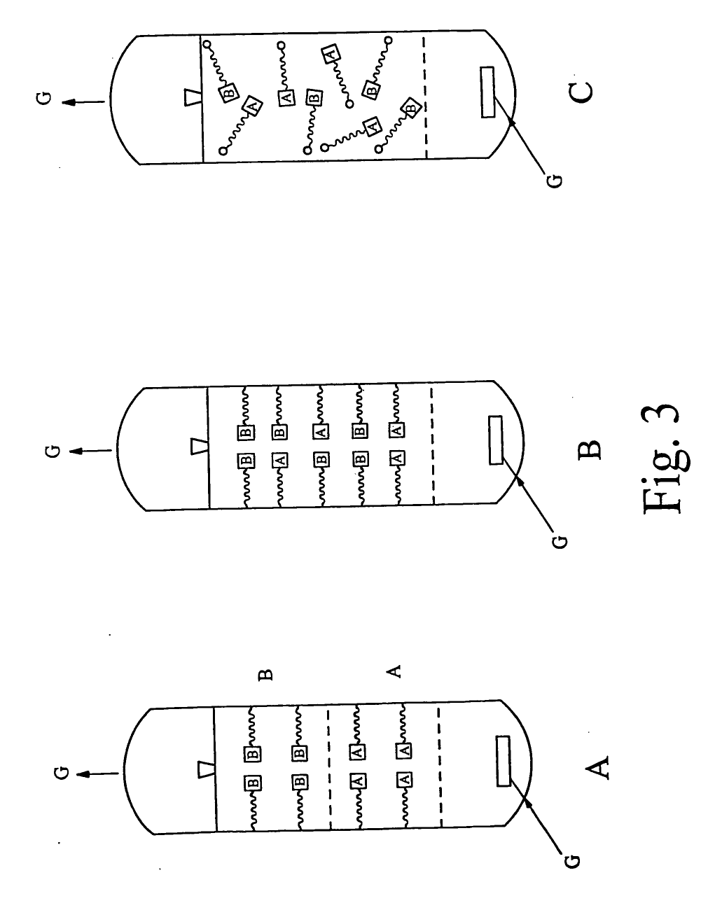

By way of example, FIG. 3 schematically illustrates some embodiments of bioreactors according to the invention.

FIG. 3A shows a bioreactor of which the chemically inert walls are modified by coupling of an immobilized enzyme of type A and an immobilized enzyme of type B which are arranged in different successive reaction zones. The letter G denotes the gas stream.

FIG. 3B shows a bioreactor of which the chemically inert walls are modified by coupling of an immobilized enzyme of type A and an immobilized enzyme of type B which are arranged in a single reaction zone.

FIG. 3C shows a bioreactor in the form of a fixed-bed reactor onto whose carrier material or bulk material immobilized enzymes of type A and type B arranged in a single reaction zone are coupled.

There are many other variants which the expert will readily consider on reading the present specification without departing from the scope of the invention.

The enzymatic system according to the invention may also be used in chromatographic systems, more particularly in chromatographic columns. This may be done for synthesis purposes (for example carrying out enzyme-catalyzed reactions in a chromatographic column) or even for analysis purposes (for example in analytical column chromatography). In this case, the immobilized enzymes of the enzymatic system may be bound, for example, to the stationary carrier material or bulk material, more particularly the stationary column material, of the chromatographic system. Further particulars can be found in the above-cited German patent application DE 101 18 553.7 of which the entire disclosure is hereby included by reference in the present application.

The use of the enzymatic system according to the invention has the advantage that even those processes which take place enzymatically in several stages can be enzyme-catalyzed using the system according to the invention, i.e. processes where the molecules, more particularly biomolecules or technical molecules, undergo enzyme-catalyzed degradation or are reacted to end products in several stages and with various enzymes.

In addition, the use of the enzymatic system according to the invention also enables several substances to be reacted in parallel by different enzymes or by enzyme (degradation) chains which, in turn, provides for example for the parallel analytical determination of several substances alongside one another using a single measuring cell containing an enzyme degradation chain of various enzymes or for the sequential degradation of a starting material (analyte) in several successive measuring cells with simultaneous electroanalysis in one and/or more measuring cells.

Finally, the use of the enzymatic system according to the invention also has the advantage that, on the one hand, the enzymes can be re-used by virtue of their immobilization and, on the other hand, are easy to remove after they have been used (for example after synthesis in the bioreactor, for example by draining off the reaction mixture). In this way, the enzymes can be efficiently and inexpensively used in high local concentrations and in continuous throughflow. However, the substrate specificity, the specificity of the reaction and the reactivity of the enzymes are not lost in the process.

Accordingly, the concept of the present invention consists in combining various enzymes with one another in such a way that molecules (for example technical molecules or biomolecules) can be degraded in this way. This may be utilized, for example, for analytical purposes in order to detect the degraded molecules through certain parameters as described above (for example changes in pH, oxygen consumption/production, etc.). With regard to bioreactors, this means that it is also possible to react one or more substances at the same time or in sequence.

Under the concept according to the invention, the production of a substance and the monitoring of reactions with sensors which measure oxygen or H₂O₂, for example, are identical and hence also provide for efficient reaction control.

Other embodiments and variations of the present invention will be readily apparent and practicable to the expert on reading the present specification without departing from the scope of the invention.

The following Examples are intended to illustrate the invention without limiting it in any way.

### EXAMPLES

### Enzymatic Degradation Chains for Alkyl Polyglucosides (APG®) in Biosensors According to the Invention

An enzymatic degradation chain consisting of amyloglucosidase and glucoseoxidase and, optionally, mutarotase and/or α-glucosidase (maltase) was developed for APG® (alkyl polyglucosides, a group of nonionic surfactants marketed by Henkel).

To produce the biosensor according to the invention, the above-mentioned enzymes were first immobilized.

Immobilization was carried out by applying the above-mentioned enzymes to a cellulose acetate membrane or to a Teflon® membrane, as described in German patent application DE 101 18 553.7. To this end, the membrane was modified or activated by plasmachemical methods known per se, i.e. functionalized in a high-frequency plasma under conditions known per se, suitable enzyme-reactive functional groups, more particularly amino and/or carboxyl groups, thus being bound to the chemically inert membrane surface. The enzymes were then bound to those groups by methods known per se.

Alternatively, immobilization may also be carried out by known methods, for example by introduction between films (for example of cellulose acetate) or in polymeric matrixes (for example PVC, gels, graphites, zeolites, etc.).

APG® were then detected or electroanalytically determined with this enzymatic degradation chain.

1. Alkyl Polyglycosides

For about 10 years, there has been a considerable increase in the production of alkyl polyglycosides. Phosphate-free, they are added as surface-active neutral surfactants to cosmetic preparations or detergents. One to seven glucose units—decreasing drastically in frequency from mono- to heptaglycosides—are glycosidically linked to a generally long-chain alcohol, such as dodecanol or tetradecanol.

Since, on the basis of enzymes, all the biochemical information for recognizing the analyte can be incorporated in a membrane system, bioelectrochemical membrane electrodes are a particularly attractive measuring principle for alkyl polyglycosides.

Of the various molecularly selective APG® membranes with immobilized enzymes designed hitherto, the principles according to the invention are illustrated in the following with reference to eleven membrane systems.

The water-soluble alkyl polyglycoside APG® 220 (C₈₋₁₀ alkyl polyglycoside) from Henkel KGaA, Düsseldorf, was available for analysis. All measuring solutions were phosphate-buffered to a pH of 5.0.

2. Construction of Molecularly Selective APG® Sensors

2.1. APG® Sensors

The sensors according to the invention are integrated into a throughflow measuring system, the enzyme membranes for the APG® substrate reaction forming the roof of a throughflow chamber with radial inflow and outflow channels flattened off in the manner of a trough. The tangential flow to the enzyme membrane is effected by drawing in of the APG®-containing solutions under suction by a roller pump following the measuring cell, measuring and calibrating solutions being fed in after the removal of air bubbles.

2.1.1. H₂O₂-Sensitive Enzymatic APG® Sensors

2.1.1.1. Modular Measuring System

The sensors in question are amperometric APG® biosensors with H₂O₂ detectors based on disk electrodes with no internal electrolyte of a Pt measuring anode and an Ag reference cathode each 2 mm in diameter in two separate serially arranged throughflow chambers connected by a flexible tube. The measuring anode and reference cathode are made in the form of spark plugs and are sealingly screwed against the edges of the throughflow chambers. Only the end faces of the noble metal electrodes are freely accessible and are electroanalytically active. The membrane systems with a diameter of 5 mm—each cavity-protected—are in direct contact with the Pt measuring anode.

In special embodiments of the enzyme membranes, the surface is made larger by cylindrical lining of the cavity optionally by coating the Pt surface (SBC-1412-APG®). Convex electrode surfaces in conjunction with thin enzyme membranes promote the rapidity of adjustment.

For a special form of application, the measuring anodes and reference cathodes described herein were installed in the cover and base of plastic vessels (SBC-1420-APG®) for stationary measurement, for example for a customer services' measuring box. In another embodiment, these measuring system components were integrated into the side walls of an acrylic glass tube fitted with inflow and outflow taps at its upper and lower ends, respectively.

2.1.1.2. Measuring System with an Anodic Window

The Pt measuring anode with a cavity-protected enzyme membrane 5 mm in diameter has direct access to the measuring solution through an anodic window while the rod-like Ag reference cathode (diameter 1 mm) is accommodated in a side space of the measuring cell and communicates with the Pt measuring anode through an internal electrolyte.

2.1.2. O₂-Sensitive Enzymatic APG® Sensors

These APG® sensors consist of a Pt cathode with a membrane system (cf. 2.1.2.1. and 2.1.2.2.) based on PTFE and immobilized enzymes. The Ag/AgCl reference anode is again accommodated in a side space of the measuring cell as in 2.1.1.2. and communicates via a buffered internal electrolyte with the Pt measuring electrode in the cathodic window which is closed by the PTFE membrane in ion-impermeable but gas-permeable manner so that O₂ can act as transducer between the immobilized enzymes and the Pt cathode.

2.1.2.1. Membrane System with a Chemically Unmodified PTFE Membrane

On the electrode side, the membrane system has a chemically unmodified PTFE membrane on the measuring solution side of which lie two cellulose diacetate dialysis membranes between which the enzymes—protected against bacterial degradation—are immobilized and/or covalently crosslinked on a strip of cellulose fibers by adsorption and/or ionic bonding.

2.1.2.2. Membrane System with a Chemically Modified PTFE Membrane

The bifunctional reagent glutardialdehyde couples covalently onto the PTFE membranes aminated in a high-frequency plasma with simultaneous crosslinking of APG®-reacting enzymes.

3. APG®-Selective Enzyme Membranes

3.1. Bienzyme Membranes

The enzymatic attack on the α-1,6-glucosidic bonds of APG® 220 by amyloglucosidase gives glucose:

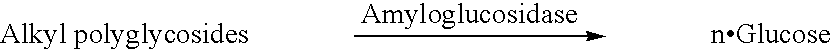

of which the β-form is further reacted by glucoseoxidase (GOD):

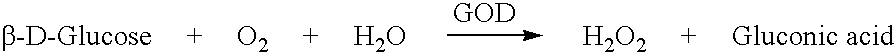

Through the reaction, the amperometric measurement can take place at the end of the enzyme degradation chain via the consumption of O₂ by oxygen electrodes or the formation of H₂O₂ with hydrogen peroxide detectors.

FIG. 1 shows a U/I diagram for bienzyme membranes for alkyl polygycoside biosensors with hydrogen peroxide detectors (t=20.0° C.)

| SBS No. | Amyloglucosidase | Glucoseoxidase |
|---|---|---|
| 1417 | 6.7 U | 6.7 U |
| 1419 | 26.7 U | 26.7 U |
| 1408 | 40.0 U | 40.0 U |

For the APG® sensors with hydrogen peroxide detectors of the 2.1.1.1. type with the SBC-1408, SBC-1417 and SBC-1419 membranes, FIG. 1 graphically illustrates the connection between the enzyme concentrationₜₒₜₐₗ in U/membrane, the current as measured at 7500 ppm APG® 220 (marking points) and the settling time.

The bienzyme membranes contain the two enzymes amyloglucosidase and glucoseoxidase in the same concentration based on units of which the total concentration is shown in the graph of FIG. 1. The enzymes are present in the membrane systems in glutardialdehyde-crosslinked form.

Of the three measuring systems with bienzyme membranes shown in FIG. 1, SBC-1419-APG® has the highest analytical resolving power.

An increase in the enzyme concentration (SBC-1408-APG®) lengthens the intramembranal diffusion pathways. Thickening of the membrane results in an increase in the settling time. At the same time, resolving power is reduced. It appears worth discussing that this could be caused inter alia by the greater decomposition of H₂O₂ on the longer diffusion pathway to the Pt measuring anode with an additional loss by diffusion through the membrane surface.

As an expression of a lower enzyme concentration (SBC-1417-APG®), there is a reduction in the substrate conversion and hence the measuring current. The faster settling times are a result of the shorter intramembranal diffusion pathways (thinning of the membrane).

In principle, the bienzyme membrane SBC-1419-APG® is also suitable for use in the H₂O₂-sensitive enzymatic measuring system with anodic window (2.1.1.2.), as is the trienzyme membrane described in the following.

3.2. Trienzyme Membranes

In order further to increase the sensitivity of the APG® sensor, the trienzyme membrane SBC-1425-APG® was developed by additional covalent bonding or crosslinking of mutarotase with glutaraldehyde (Table 1).

TABLE 1
Bi- and trienzyme membranes for alkyl polyglycoside
biosensors with hydrogen peroxide detectors

| SBC No. | Amyloglucosidase | Glucoseoxidase | Mutarotase |
|---|---|---|---|
| 1419 | 26.7 U | 26.7 U | — |
| 1425 | 22.9 U | 22.9 U | 142.9 U |

GOD is known to react highly selectively with β-D-glucose whereas the α-form is not reacted by this enzyme.

In their work on “Alkylpolyglycoside—Eigenschaften und Anwendungen einer neuen Tensidklasse” in Angew. Chem. 110 (1998), pages 1395 to 1412, von Rybinski, W. and Hill, K. report that, in the industrial process, the α-anomers and β-anomers are in a ratio of approximately 2:1 to one another. However, since almost conversely 36% of α-D-glucose and 64% of β-D-glucose are present from freshly prepared α-D-glucose about 2 hours after establishment of the mutarotation equilibrium, it had been expected that, with the aid of the additionally incorporated mutarotase:

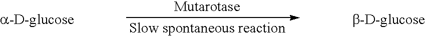

a higher intramembranal substrate supply for the glucoseoxidase would be able to be induced in an accelerated reaction on the basis of the assumption that the ratio between the α- and β-anomers in the product APG® 220 is similar to that encountered in the industrial process.

Using modular measuring systems (cf. 2.1.1.1.) with H₂O₂ detectors, it was possible to show that, in relation to the bienzyme membrane SBC-1419-APG®, sensor sensitivity was increased by ca. 30% through the additional presence of the mutarotase in the trienzyme membrane SBC-1425-APG® although the two other enzymes were actually present in the trienzyme membrane system in slightly lower enzymatic concentrations (cf. Table 1).

3.3. Tetraenzyme Membranes

The first molecularly selective membrane electrodes for determining APG® were developed on the basis of four enzymes and, besides amyloglucosidase, glucoseoxidase and mutarotase, also contained α-glucosidase with a view to possibly releasing the fatty alcohol from its glycosidic linkage (Table 2).

TABLE 2
Tetraenzyme membranes for O₂-sensitive
APG ® multienzyme electrodes

| SBC | | | | α- |
|---|---|---|---|---|
| No. | Amyloglucosidase | Glucoseoxidase | Mutarotase | Glucosidase |
| 1400 | 52.0 U | 40.0 U | 1056.0 U | 20.8 U |
| 1402 | 150.0 U | 120.0 U | 1000.0 U | 20.8 U |

Unfortunately, α-1,6-glucosidic bonds are only slowly reacted by the enzyme also known as maltase. There is no attack on β-glucosidic bonds. By contrast, α-1,4-glucosidic bonds are the preferred point of attack.

The four enzymes were immobilized by adsorption and/or ionic bonding to a strip of cellulose fibers between two cellulose acetate dialysis membranes. The enzyme membrane was covered with a chemically unmodified PTFE membrane, clamped to an acrylic glass ring by an O ring and, finally, positioned via an internal electrolyte film in front of the Pt cathode of the O₂ sensor which was completed by an Ag/AgCl reference cathode with the common internal electrolyte to form the measuring cell: O₂-sensitive APG® multienzyme membrane electrodes.

3.4. APG® Biosensors with Enzymes on Aminated PTFE Membranes

Amyloglucosidase and glucoseoxidase were covalently bonded by the bifunctional reagent glutardialdehyde to chemically modified PTFE membranes in aminated form (2.1.2.2.) for oxygen electrodes (2.1.2.). In other layers, these enzymes were crosslinked and coupled to the previous layer. Among the enzyme membranes covered by Table 3, the APG® sensor with 10.) SBC-1322-APG®-HDKS5-No. 1 is distinguished not only by high measured value stability, but also by settling times of only 3 to 6 minutes. This is attributable to a particularly streamlined geometry, tangential flow to the membrane system as a whole and the absence of enzyme-encapsulating dialysis membranes.

TABLE 3
Aminated PTFE membranes with covalently
bonded enzymes for O₂ detectors of
molecularly selective APG ® sensors

| Laboratory Code | Amyloglucosidase | GOD |
|---|---|---|
| 7.) SBC-1322-APG ®-HDKS3-No. 1 | 60.0 U | 120.0 U |
| 8.) SBC-1322-APG ®-HDKS4-No. 1 | 140.0 U | 120.0 U |
| 10.) SBC-1322-APG ®-HDKS5-No. 1 | 40.0 U | 40.0 U |

4. Results

4.1. Settling Time

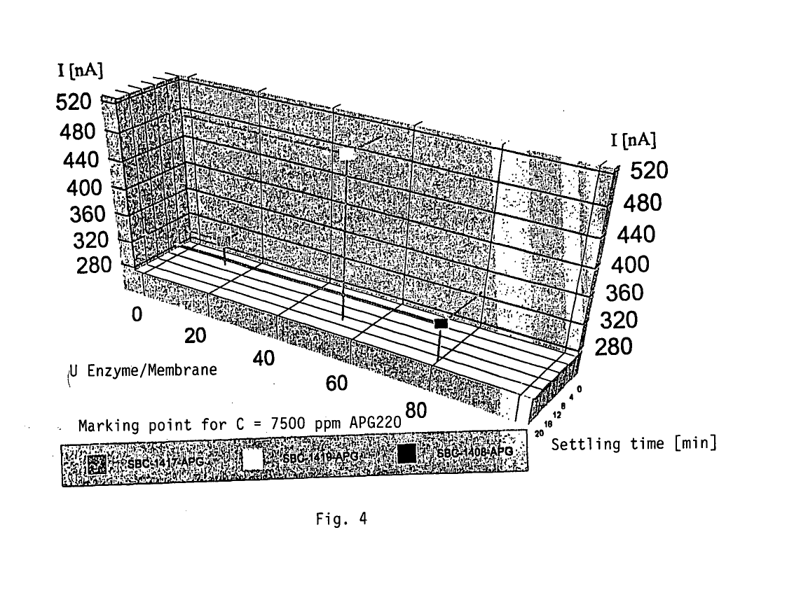

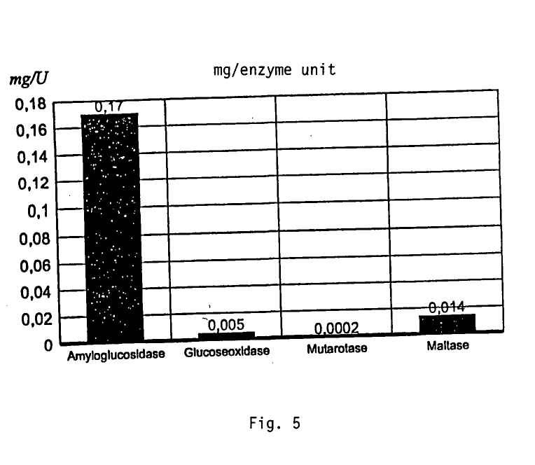

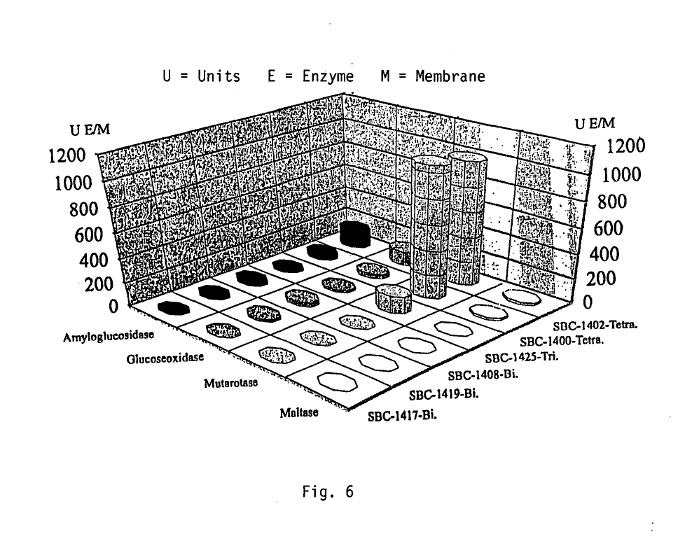

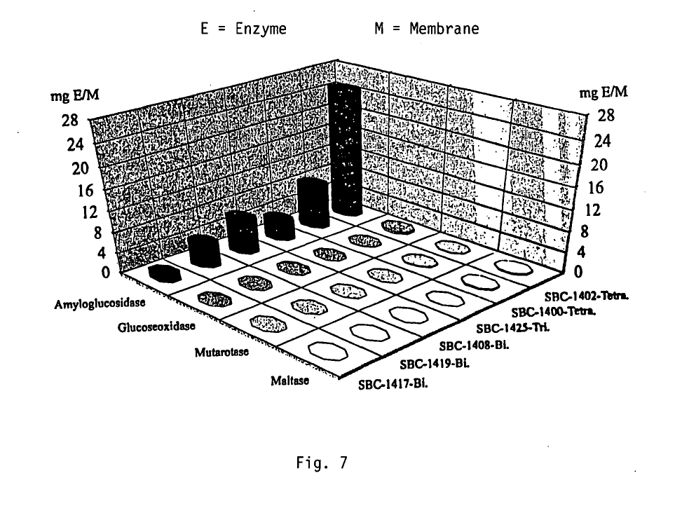

Besides the number of changes of the individual enzymes and streamlined positioning of the membrane systems, the concentrations of immobilized enzymes influence the settling time of the APG® sensor membranes through the concentration-related length of the diffusion pathways. According to FIG. 2, the ratio of weight (mg) to substrate reaction (unit) of the enzymes immobilized in the APG® sensor membranes suggests that the speed-determining step in the intramembranal diffusion process starts out from the amyloglucosidase. Comparison of FIG. 3 with FIG. 4 confirms this connection (cf. also FIG. 1). However, this also accounts for the creeping settling behavior of SBC-1402. By contrast, other APG®-selective membrane electrodes with O₂ detectors and immobilized enzymes on aminated PTFE membranes—again with tangential flow and no dialysis membranes—show short settling times of a few minutes: cf. 10.) SBC-1322-APG®-HDKS5-No. 1 under 3.4 and Table 3, whereas APG® sensors with hydrogen peroxide detectors offer superior sensitivity or greater resolving power (cf. FIG. 1).

FIG. 2 shows the ratio of weight (mg) to substrate reaction (U) of the enzymes used in the APG® sensor membranes (1 unit=1 μmol substrate reaction/min.).

FIG. 3 shows the enzyme concentrations in U/APG®-selective biosensor membrane.

In FIGS. 3 and 4, the columns for mutarotase and maltase (α-glucosidase) of the “Bi.” membranes (“Bi.”=abbreviation for bienzyme membrane) are put at zero; for SBC-1425-Tri. (Tri.=trienzyme membrane), this applies correspondingly to the α-glucosidase (maltase) only.

FIG. 4 shows the enzyme concentrations in mg/APG®-selective biosensor membrane.

Raising the temperature of the measuring medium can be expected to shorten the settling times of APG® sensors. In order safely to rule out heat-induced denaturing of the enzymes, an increase in temperature above 40° C. was avoided. For the trienzyme membrane SBC-1425-APG®, the factor 1.3 was determined for a temperature increase of 10° C. on H₂O₂ detectors of the modular throughflow measuring system constructed in accordance with 2.1.1.1., which also suggests that the physical process of diffusion is critical as the speed-determining step for the settling times of this APG® sensor. This is because it is known that, under the RGT rule, the speed of a chemical reaction increases by a factor of 2 to 4 when the temperature rises by 10° C. Accordingly, this chemical reaction would appear to be overshadowed by the intramembranal diffusion processes, so that a factor of only about 1.2 can be expected.

4.2. Period of Operation

Since the focus of attention had hitherto been the development of membrane-chemical principles, the continuous long-term perfusions of the APG® sensors with phosphate buffer of pH 5.0 and interim measurements in APG®-containing solutions were terminated after a few weeks.

4.3. Molecular Selectivity

With APG® sensors, detector-related cross-sensitivities basically have to be taken into account in addition to the substrate selectivity of the enzymes. In O₂ detectors, such cross sensitivities cause interfacially active or gaseous, cathodically reactable substances which permeate the PTFE membrane, such as for example the halogenated hydrocarbons chloroform (trichloromethane) or the inhalation narcotic agent halothane (1,1,1-trifluoro-2-bromo-2-chloroethane).

Hydrogen peroxide detectors, in their capacity as measuring anodes, also react other anodically oxidizable substances. The glucose interference current observed at a polarization voltage of +950 mV measuring against reference electrode on freshly polished platinum anodes in phosphate buffer solutions with a pH of 7.04 containing 5.55 mmol/l glucose showed an e-functional drastic reduction within two weeks and became almost negligible thereafter.

For a concentration of 7500 mg/l APG® 220 in solutions phosphate-buffered to a pH of 5.0, non-freshly polished Pt measuring anodes in a modular measuring system according to 2.1.1.1. yielded only 1.33% of the measuring current in relation to one coated with the biezyme membrane SBC-1419. This slight additional current directly inducible at the detector by the APG® molecules as a function of concentration cannot be regarded as an interference signal because it acts synergistically to the measuring current triggered by the enzyme membrane and can be calibrated.

5. Electronic Components and Computer-Assisted Measured Data Evaluation

The nanoampere transducer supplies a polarization voltage “plateau-adapted” to the amperometric sensor and has a transducer constant of 1 mV/nA. For O₂-sensitive enzymatic measuring systems, the polarization voltage amounts to −750 mV measuring against reference electrode and, for H₂O₂-sensitive enzymatic measuring systems with anodic oxidation, to +950 mV measuring against reference electrode.

The 21-bit AD transducer converts the electrical d.c. voltages supplied by the nanoampere transducer after current/voltage transformation into corresponding digital signals which are transmitted via a serial interface (RS 232) to an IBM-compatible computer in order to carry out a computer-assisted measured data evaluation from the measured currents. The measuring curve with a measured value in about 2 seconds can thus be continuously followed on the screen.

Besides alkyl polyglucosides and other surfactants, further possible applications for the biosensors include, for example, the determination of polyaspartic acid and fatty alcohol derivatives.

Substances in the field of reactors may be, for example, substances which are used in situ by the consumer, including for example peroxides or surfactants, lubricants or high-quality chemical specialities.

1-42. (cancelled).

43. A process for reacting a target molecule with at least two different enzymes comprising:
(a) providing at least one target molecule;
(b) providing at least one reaction zone;
(c) providing at least two different enzymes, present in the reaction zone, wherein at least one of the enzymes is immobilized therein;
(d) introducing the target molecule into the reaction zone; and
(e) reacting the target molecule, with the enzymes, in the reaction zone.

44. The process of claim 43 wherein the enzymes are arranged in a predetermined sequence within the reaction zone.

45. The process of claim 43 further comprising providing multiple reaction zones, each of which contains at least one enzyme.

46. The process of claim 43 wherein at least one of the enzymes is immobilized within the reaction zone by crosslinking.

47. The process of claim 43 wherein at least one of the enzymes is immobilized within the reaction zone by enclosing the enzyme within a semipermeable membrane or between at least two semipermeable membranes.

48. The process of claim 47 wherein the semipermeable membrane is selected from the group consisting of polytetrafluroethylene, silicone rubber, and combinations thereof.

49. The process of claim 43 wherein at least one of the enzymes is immobilized within the reaction zone by binding the enzyme to a surface of a chemically inert carrier substrate.

50. The process of claim 49 wherein the surface of the carrier substrate is plasmachemically activated.

51. The process of claim 49 wherein the surface of the carrier substrate is functionalized with at least one functional group reactive to at least one of the enzymes.

52. The process of claim 49 wherein the surface of the carrier substrate has been both plasmachemically activated and functionalized with at least one functional group reactive to at least one of the enzymes.

53. The process of claim 43 wherein the target molecule is selected from the group consisting of a naturally occurring molecule, a non-naturally occurring molecule, and mixtures thereof.

54. The process of claim 46 wherein the crosslinking is performed using glutardialdehyde.

55. The process of claim 43 wherein the reaction zone is a biosensor.

56. The process of claim 43 wherein the reaction zone is a bioreactor.

57. The process of claim 43 wherein the reaction zone is a chromatographic column.

58. An apparatus for reacting a target molecule with at least two different enzymes comprising:
(a) at least one reaction zone; and
(b) at least two different enzymes, present in the reaction zone, wherein at least one of the enzymes is immobilized therein whereby, in operation, the target molecule is introduced into the reaction zone at which time it reacts with the enzymes present therein.

59. The apparatus of claim 58 wherein the enzymes are arranged in a predetermined sequence within the reaction zone.

60. The apparatus of claim 58 further comprising multiple reaction zones, each of which contains at least one enzyme.

61. The apparatus of claim 58 wherein at least one of the enzymes is immobilized within the reaction zone by crosslinking.

62. The apparatus of claim 58 wherein at least one of the enzymes is immobilized within the reaction zone by enclosing the enzyme within a semipermeable membrane or between at least two semipermeable membranes.

63. The apparatus of claim 62 wherein the semipermeable membrane is selected from the group consisting of polytetrafluroethylene, silicone rubber, and combinations thereof.

64. The apparatus of claim 58 wherein at least one of the enzymes is immobilized within the reaction zone by binding the enzyme to a surface of a chemically inert carrier substrate.

65. The apparatus of claim 64 wherein the surface of the carrier substrate is plasmachemically activated.

66. The apparatus of claim 64 wherein the surface of the carrier substrate is functionalized with at least one functional group reactive to at least one of the enzymes.

67. The apparatus of claim 64 wherein the surface of the carrier substrate has been both plasmachemically activated and functionalized with at least one functional group reactive to at least one of the enzymes.

68. The apparatus of claim 58 wherein the target molecule is selected from the group consisting of a naturally occurring molecule, a non-naturally occurring molecule, and mixtures thereof.

69. The apparatus of claim 61 wherein the crosslinking is performed using glutardialdehyde.

70. The apparatus of claim 58 wherein the apparatus is a biosensor.

71. The apparatus of claim 58 wherein the apparatus is a bioreactor.

72. The apparatus of claim 58 wherein the apparatus is a chromatographic column.

73. A process for reacting a target molecule with at least two different enzymes comprising:
(a) providing at least one target molecule;
(b) providing at least one reaction zone;
(c) providing at least two different enzymes, present in the reaction zone, wherein at least one of the enzymes is immobilized therein;
(d) introducing the target molecule into the reaction zone; and
(e) reacting the target molecule, with the enzymes, in the reaction zone, and wherein the at least one enzyme is immobilized in the reaction zone by a process involving the steps of:
(i) activating a chemically inert carrier surface by binding at least one suitable functional group, reactive to the enzymes, directly to the chemically inert carrier surface using a plasmachemical method;
(ii) binding the enzyme(s) to be immobilized, to the carrier surface activated in step (i); and
(iii) optionally, crosslinking the enzymes bound to the carrier surface in step (ii).

74. The process of claim 73 wherein process steps (ii) and (iii) are carried out simultaneously.

75. The process of claim 73 wherein the functional group is selected from the group consisting of a carboxyl group, an amino group, a hydroxy group, a thio group, and combinations thereof.

76. The process of claim 75 wherein the functional group protonated or deprotonated.

77. An apparatus for reacting a target molecule with at least two different enzymes comprising:
(a) at least one reaction zone; and
(b) at least two different enzymes, present in the reaction zone, wherein at least one of the enzymes is immobilized therein, and wherein the at least one enzyme is immobilized in the reaction zone by a process involving the steps of:
(i) activating a chemically inert carrier surface by binding at least one suitable functional group, reactive to the enzymes, directly to the chemically inert carrier surface using a plasmachemical method;
(ii) binding the enzyme(s) to be immobilized, to the carrier surface activated in step (i); and
(iii) optionally, crosslinking the enzymes bound to the carrier surface in step (ii), whereby, in operation, the target molecule is introduced into the reaction zone at which time it reacts with the enzymes present therein.

78. The apparatus of claim 77 wherein process steps (ii) and (iii) are carried out simultaneously.

79. The apparatus of claim 77 wherein the functional group is selected from the group consisting of a carboxyl group, an amino group, a hydroxy group, a thio group, and combinations thereof.

80. The apparatus of claim 79 wherein the functional group protonated or deprotonated.
